(19) United States
(12) Patent Application Publication
(10) Pub. No.: US 2021/0137071 A1
(43) Pub. Date: May 13, 2021

(54) RECIRCULATION SYSTEM AND METHOD

(71) Applicant: Applied LifeSciences and Systems LLC, Raleigh, NC (US)

(72) Inventor: Stephen WOLFE, Raleigh, NC (US)

(73) Assignee: Applied LifeSciences and Systems LLC, Raleigh, NC (US)

(21) Appl. No.: 17/259,002

(22) PCT Filed: Jul. 25, 2019
(86) PCT No.: PCT/US19/43381

§ 371 Date: Jan. 8, 2021

Related U.S. Application Data
(60) Provisional application No. 62/703,247, filed on Jul. 25, 2018.

(51) Int. Cl. A01K 13/00; A01K 45/00; A61D 7/00; A61M 5/30; A61M 11/00; A61M 39/24

(52) U.S. Cl. CPC A01K 13/003; A01K 45/00; A61D 7/00; A01K 61/13; A61M 11/006; A61M 39/24; A61M 5/30

(57) ABSTRACT

A system for delivery of a fluid to an animal is provided in the present disclosure. The system includes a reservoir to hold a volume of fluid, delivery outlet, recirculating loop, and valve in fluid communication between the reservoir and delivery outlet, the valve having an inlet to receive fluid from the reservoir, a first outlet in fluid communication with the delivery outlet, and a second outlet in fluid communication with a return conduit to the reservoir. The system further includes control means for opening and closing the first and second valve outlets, and a pump to pump fluid from the reservoir to the valve and through the reservoir return conduit to the reservoir. When the control means opens the first outlet, the fluid flows from the reservoir to the delivery outlet. When the control means opens the second valve outlet, the fluid flows through the return conduit to the reservoir.

### PRIORITY

This application claims priority from U.S. provisional patent application Ser. No. 62/703,247, filed Jul. 25, 2018. The contents of which are incorporated herein in its entirety.

### CROSS REFERENCE TO RELATED APPLICATIONS

This application references PCT International Application Number PCT/US2019/043381, titled RECIRCULATION SYSTEM AND METHOD and filed Jul. 25, 2019.

### BACKGROUND

Bacterial, viral and fungal infections and other diseases are often prevented or treated through vaccination, or delivery of a drug to a subject. In all animals, and in particular vertebrates such as mammals or fish and invertebrates such as crustaceans, the delivery of vaccines, biologics and other medicine is often utilized to prevent disease, death or to maintain overall good health. In many livestock and fish operations it is a challenge to ensure that all animals have been effectively treated.

Some vaccines, biologics and other medicine have particulates in solution. In particular, some vaccines have live cultures in solution. The variation in the size of the particulate in solution can affect delivery, due to settling or clogging of particulate leading to a lack of uniformity during delivery. Some subjects may receive more or less particulate if the particulate is not suspended in solution uniformly.

Turning now to the poultry industry, there are several current methods in which fertilized eggs or chickens are treated with medicine. These include:
1) Automated Vaccination in the hatchery performed in ovo (within the egg) on day 18 or19;
2) Manual Vaccination in the hatchery performed post-hatch;
3) Vaccination/Medication added to the feed or water in a grow out farm; and
4) Vaccination/Medication sprayed on the chicks either manually or by mass-sprayers.

In addition, a new system and method for delivering vaccines and other substances to animals has been developed. The system and method are disclosed in PCT/US2016/061548 which discloses in part a system for automatically delivering a substance to an animal, and the contents of which are incorporated herein in their entirety. The system includes a series of conveyor belts and other moving platforms and the like to separate each animal, in particular day-old chicks. Once the chicks are separated into single file formations, the system is able to deliver one or more doses of vaccine, medicine, biologic, supplement or other substance to the individual animal. The system includes a nozzle for delivering substance to the mucosal area of a chick's face, such as the eye, nasal passage, or mouth (if the chick's beak is open). The nozzle may be air atomizing or hydraulic.

Substances such as a vaccine, medicine, biologic or supplement are often delivered in solution. The active ingredient in a vaccine, medicine, biologic or supplement is typically diluted in a solution of water, saline or the like for easier and more effective delivery. For vaccines, the live cultures, including oocysts, are suspended within solution for effective delivery. One such type of oocyst vaccine is described in detail in PCT/US19/41178 entitled “Systems and Methods of Preparing and Delivering Oocyst Solutions,” which is incorporated herein by reference in its entirety.

In some situations, difficulties may arise with the use of a nozzle in delivering a solution because the active ingredient needs to stay suspended in the solution for effective uniform delivery of the active ingredient. The live cultures cannot be allowed to settle to the bottom of reservoirs, tanks, vaccine bags, vaccine lines, nozzles or other containers, because it will lead to non-uniform delivery. Non-uniform vaccination occurs when some animals get high doses of the active ingredient while others get none.

In the past, nozzles or spray heads have been primed to prevent non-uniform delivery thereon. However, this method is wasteful of the substance, or is often ineffective if the system operators forget to prime the spray head. When this happens, many animals are receiving no active vaccine particles. This will lead to poor performance and outbreaks when these birds are exposed to pathogens during grow out. In addition, the amount of vaccine wasted through priming of the nozzles costs the hatcheries significant amounts of money annually.

The embodiments described herein provide a system and method for delivering substance to an animal where the spray head does not require priming every time there is a break in the process. This system enables the substance to remain suspended in solution while the system is either active or at rest.

### SUMMARY

The embodiments described herein are directed to a system and method for delivery of a fluid to an animal. The system has a reservoir to hold a volume of fluid, a delivery outlet, and a recirculating loop. The system also includes a valve in fluid communication between the reservoir and delivery outlet, the valve has an inlet to receive fluid from the reservoir, a first outlet in fluid communication with the delivery outlet, and a second outlet in fluid communication within the recirculating loop. The system further includes a control means for controlling the position of the valve outlet, a reservoir pump to pump fluid from the reservoir to the valve, and a recirculation pump to pump fluid within the recirculation loop. When the control means opens the valve outlet to the first outlet, the fluid flows from the reservoir to the delivery outlet, and when the control means closes the first outlet and opens the valve outlet to the second outlet, the fluid flows within the recirculation loop.

### DESCRIPTION OF THE DRAWINGS

Having thus described various embodiments of the present disclosure in general terms, reference will now be made to the accompanying drawings, which are not drawn to scale and do not include all components of the system, and wherein:

FIG. 1 is a schematic view of the system of the first embodiment;

FIG. 2A is a sectional view of a portion of the nozzle and valve of the first embodiment in delivery mode;

FIG. 2B is a sectional view of the nozzle and valve of the first embodiment in recirculation mode; and

FIG. 3 is a schematic view of a system of the second embodiment.

### DETAILED DESCRIPTION

The present disclosure is directed to system and method for delivery of a fluid to an animal. Various aspects of the present disclosure will be described more fully hereinafter with reference to the accompanying drawings, in which some, but not all aspects of the disclosure are shown. Indeed, this disclosure may be embodied in many different forms and should not be construed as limited to the aspects set forth herein.

One embodiment is directed to the delivery of a substance to chicken hatchlings after they have been separated from their shells and prior to departure from the hatchery. In addition, methods and systems according to aspects of the present disclosure relating to chicks may be used with any type of poultry including, but not limited to, chicken, turkey, duck, geese, quail, pheasant, ostrich, exotic birds, and the like.

The system and methods of the present embodiment are used in connection with the delivery of a fluid to an animal. In the present embodiment, the system is used to spray a fluid into the mucosa (eyes, nasal passage or mouth) of a day-old chick. This is accomplished by separating the chicks singularly and positioning them in front of a spray head, which is part of the first embodiment described below. The fluid is delivered through the spray head and into the mucosa on the face of a day-old chick.

FIG. 1 illustrates a simplified schematic view of the overall system of the first embodiment 10. The system includes a reservoir 12 filled with fluid 14. The fluid 14 may be a vaccine, medicine, biologic or supplement diluted in solution. The diluent may be water, saline or the like.

The reservoir 12 includes a fluid inlet or opening 16 into which fluid 14 is poured prior to use. The fluid inlet has a cap or cover 18 which is securely fastened to the reservoir 12 during use. The reservoir 12 also has a pressurized air inlet 60. The pressurized air inlet 60 is connected to a pressurized air supply 20 by means of a first pressurized air conduit 22. A first pressure regulator 24 is located along the first pressurized air conduit 22 to control the pressure along the first pressurized conduit.

The reservoir 12 further includes a fluid outlet 26. The fluid outlet 26 is connected to a first fluid conduit 28. The first fluid conduit 28 connects to a recirculation pump inlet 30 on a recirculation pump 32. The recirculation pump 32 has a pump fluid outlet 34. The pump fluid outlet 34 is connected to a second fluid conduit 36. The second fluid conduit 36 connects to a valve fluid inlet 38. The valve fluid inlet 38 is mounted on a three-way valve 40. The valve 40, as shown in FIGS. 2A and 2B, has a fluid inlet 56, and two outlets: a by-pass port 46, and an outlet port 44. The by-pass port 46 is in fluid communication with the by-pass outlet 42 which receives fluid from the valve 40 when the by-pass function is activated which will be described in more detail below. The position of the valve 40 outlets is controlled electronically by a PLC (programmable logic controller) not shown.

The by-pass outlet 42 is in fluid communication with the by-pass conduit 45 as shown in FIG. 1. The by-pass conduit 45 is in fluid communication with the by-pass inlet 47. The by-pass inlet 47 provides a fluid opening for the by-pass conduit 45 into the reservoir 12.

It should be noted that the valve 40 is capable of being in one of two positions, either a spray position as shown in FIG. 2A, or a recirculation position as shown in FIG. 2B. The spray position is when the outlet port 44 is opened and the by-pass port 46 is closed. The recirculation position is when the by-pass port 46 is opened and the outlet port 44 is closed.

The outlet port 44 of the valve is connected to a nozzle outlet conduit 48 which connects to the delivery nozzle 50 having a delivery nozzle spray head 54 as shown in FIG. 1. In an air atomizing nozzle arrangement, the nozzle spray head 54 is also connected to a pressurized air source 52. The pressurized air from the pressurized air source mixes with the fluid exiting the spray head 54 to create an atomized spray profile for delivery to an animal which will be discussed in more detail below.

In use, the pressurized air supply 20 is introduced into the reservoir 12 by way of the pressurized air inlet 60. Fluid 14, which is a solution of vaccine, medicine, biologic or supplement, suspended in a diluent, is held in the reservoir 12. Once the pressurized air supply 20 is activated the fluid 14 exits the reservoir by means of the fluid outlet 26, along the first fluid conduit 28 and into the recirculation pump inlet 30. The recirculation pump 32 moves the fluid 14 out of the pump by way of the pump fluid outlet 34 through the second fluid conduit 36 and into the valve fluid inlet 38.

During use for the delivery of fluid to an animal, the animal is positioned proximate to the nozzle spray head 50. At that time, the valve controller 46 opens outlet port 44 and this action simultaneously causes the by-pass port 42 to close. This causes fluid 14 to flow through the outlet port 44 through the nozzle outlet conduit 48 and into the nozzle spray head 50, as shown in FIG. 2A. At the nozzle spray head 50, fluid 14 is mixed with pressurized air from the pressurized air source 52. This causes the fluid 14 to become atomized and sprays the atomized fluid 14 into the mucosa of the day-old chick nearby.

When the first embodiment 10 is not being used for delivering fluid to an animal, the valve controller 46 closes outlet port 44 which simultaneously causes by-pass port 42 to open. This results in a flow of fluid 14 from the valve 40 back to the reservoir 12 by way of the outlet port 56, by-pass port 42, by-pass conduit 45, and by-pass inlet 47. Recirculation thus maintains fluid 14 as a uniform suspension, preventing settling of vaccine components. The fluid 14 will continue to recirculate in this fashion until it is needed to be delivered to an animal. At that time, the valve 40 will be redirected, as described above, to deliver fluid 14 via the nozzle spray head 54 to an animal.

It is anticipated that the types of vaccines or other substances given to chicks by spray application to the mucosa may include, but not be limited to the following: vaccinations against viruses such as Newcastle disease and infectious bronchitis virus, bacteria such as E. coli, salmonella, and campylobacter, and parasites such as coccidia.

A second embodiment 60 is shown in FIG. 3. The second embodiment 60 is similar in some respects to the first embodiment 10 in that it has a reservoir 12 and fluid 14 with particulate suspended therein. The reservoir 12 is in fluid communication with pump 32 via fluid outlet 26 and first conduit 28. The pump fluid outlet 34 connects to the by-pass conduit 45 and by-pass inlet 47. In addition, the pump outlet 34 connects to valve 62 which is in fluid communication with the delivery nozzle 50 having delivery nozzle spray head 54. The delivery nozzle operates and is controlled as described above with respect to the first embodiment 10.

In use, fluid 14 in the reservoir 12 is moved via pump 32 through the pump outlet 34 and returned to the reservoir via by-pass conduit 45 through by-pass inlet 47. When valve 62 is activated, fluid 14 is simultaneously moved through the valve into the nozzle 50 and out the nozzle spray head 54. The fluid 14 mixes with pressurized air source 52 to atomize and create a spray profile which is directed to the mucosa areas on the face of an animal.

Tests were done to determine if the recirculating pump would maintain particulate in suspension of a vaccine solution. The vaccine in solution was an Eimeria vaccine having at least three species of oocysts. These included E. maxima, E. tenella, and E. acervulina. The solution was sprayed onto a predetermined surface after the nozzle had been at rest for ten minutes. The variation in vaccine counts was detected by counting the three different species of oocysts delivered by the nozzle over time. B1 refers to nozzle 1 and B2 refers to nozzle 2. Each spray was counted, and the number of each species calculated for each sample. Counting before and after the 10 minute break detected less than a 15% deviation in the counts. The results are set forth in Table 1 below. This is close to the standard error for counting with a low number of samples and indicated that the recirculation loop to the nozzle resolved settling issues. Previously, in delivery studies without the recirculation feature, large decreases in the number of oocysts were detected after the 10-minute break.

It is also anticipated that the embodiments herein may apply to the automated delivery of substance to the mucosa of other animals and mammals, including humans. In particular, there may be certain applications that may be appropriate for automated delivery of a substance to the facial mucosa of an infant or child, or disabled person. In addition, the automated delivery system described herein may have applicability to other animals, such as livestock, companion animals, rodents and other animals raised commercially.

It is expected that many modifications and other aspects of the present disclosure set forth herein will come to mind to one skilled in the art to which this disclosure pertains having the benefit of the teachings presented in the foregoing descriptions and the associated drawings. Therefore, it is to be understood that the present disclosure is not intended to be limited to the specific aspects disclosed and that modifications and other aspects are intended to be included within the scope of the appended claims. Although specific terms are employed herein, they are used in a generic and descriptive sense only and not for purposes of limitation.

1. A system for delivery of a fluid to an animal comprising
a reservoir having a volume of fluid;
a delivery outlet;
a valve in fluid communication between the reservoir and delivery outlet, the valve having an inlet to receive fluid from the reservoir, a first outlet in fluid communication with the delivery outlet, and a second outlet in fluid communication with the reservoir by means of a reservoir return conduit;
control means for opening and closing the first and second valve outlets; and
a pump to continuously pump fluid from the reservoir to the valve and from the valve through the reservoir return conduit to the reservoir, whereby when the control means opens the first valve outlet and closes the second valve outlet, fluid flows from the reservoir to the delivery outlet, and when the control means closes the first valve outlet and opens the second valve outlet, the fluid flows through the reservoir return conduit to the reservoir.

2. The system of claim 1 wherein the delivery outlet is a nozzle.

3. They system of claim 2 wherein the nozzle is either an air atomizing spray nozzle or a hydraulic spray nozzle.

4. The system of claim 1 wherein the delivery outlet is an injector.

5. The system of claim 4 wherein the injector is needleless.

6. The system of claim 1 wherein the control means comprises a computerized control system in communication with the valve.

7. The system of claim 1 wherein the flow rate in the reservoir return conduit is about 60 milliliters per minute.

8. The system of claim 1 further comprising a pressure source in communication with the fluid reservoir and the pump.

9. The system of claim 8 wherein the pressure source is about 15 psi.

10. The system of claim 1 wherein the fluid is a vaccine, biologic, medicament, or supplement.

11. The system of claim 1 wherein the animal is a chicken.

12. A system for delivery a substance to an animal comprising:
a reservoir having a volume of fluid;
a delivery outlet;
a three-way valve having an inlet in fluid communication between the reservoir, and two outlets, the first outlet in fluid communication with the delivery outlet and the second outlet in fluid communication with a reservoir return conduit, the valve having a first position in which the first outlet is opened and the second outlet is closed to enable fluid flow from the reservoir to the delivery outlet, the valve having a second position in which the second outlet is opened and the first outlet is closed to enable fluid flow through the reservoir return conduit, the valve having a third position in which the first and second outlets are closed;
control means for controlling the positions of the valve;
a pump to continuously pump fluid from the reservoir to the valve through the reservoir return conduit and back to the reservoir.

13. The system of claim 12 wherein the delivery outlet is a nozzle.

14. The system of claim 13 wherein the nozzle is either a spray nozzle or a hydraulic nozzle.

15. The system of claim 12 wherein the delivery outlet is an injector.

16. The system of claim 15 wherein the injector is needless.

17. The system of claim 12 further comprising a pressure source in fluid communication with the fluid reservoir and the recirculation pump.

18. The system of claim 17 wherein the pressure source is about 15 psi.

19. The system of claim 12 wherein the fluid is either a vaccine, biologic, medicament or supplement.

20. A method of delivering of a fluid to an animal comprising the steps of:
providing a reservoir having a volume of fluid therein;
providing a delivery outlet;
providing a valve in fluid communication between the reservoir and delivery outlet, the valve having an inlet to receive fluid from the reservoir, a first outlet in fluid communication with the delivery outlet, and a second outlet in fluid communication with a reservoir return conduit, the reservoir return conduit in fluid communication with the reservoir; and
continuously pumping fluid from the reservoir to the valve, whereby when the first valve outlet is open, the second valve outlet is closed and fluid flows from the reservoir through the first valve outlet to the delivery outlet, and when the first valve outlet is closed the second valve outlet is open and fluid flows from the reservoir through the reservoir return conduit back to the reservoir.
